(19) United States
(12) Patent Application Publication
(10) Pub. No.: US 2014/0336391 A1
(43) Pub. Date: Nov. 13, 2014

(54) PROCESS FOR THE PREPARATION OF ASENAPINE INTERMEDIATE

(71) Applicant: RANBAXY LABORATORIES LIMITED, New Delhi, Delhi (IN)

(72) Inventors: Ramnik Sharma, Gurgaon (IN); Senkara Rao Allu, Srikakulam (IN); Ram Chander Aryan, New Delhi (IN)

(21) Appl. No.: 14/360,421

(22) PCT Filed: Nov. 7, 2012
(86) PCT No.: PCT/IB2012/056238

§ 371 Date: May 23, 2014

(30) Foreign Application Priority Data
Nov. 28, 2011 (IN) 3409/DEL/2011

(51) Int. Cl. C07D 491/044

(52) U.S. Cl. CPC C07D 491/044
USPC 548/421

(57) ABSTRACT

The present invention provides a process for the preparation of the asenapine intermediate of Formula (III) using a magnesium-methanol-acetic acid mixture.

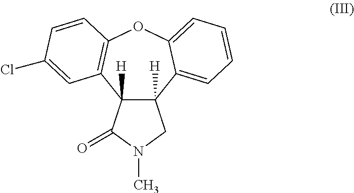

### FIELD OF THE INVENTION

The present invention provides a process for the preparation of the asenapine intermediate of Formula III using a magnesium-methanol-acetic acid mixture.

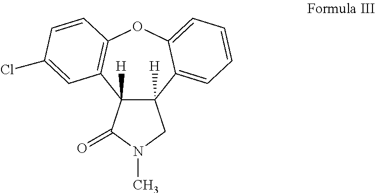

### BACKGROUND OF THE INVENTION

Asenapine and its pharmaceutically acceptable salts, including asenapine maleate, are known from U.S. Pat. No. 4,145,434. Asenapine maleate is chemically (3 aRS,12bRS)-5-Chloro-2-methyl-2,3,3a,12b-tetrahydro-1H-dibenzo[2,3:6,7]oxepino[4,5-c]pyrrole (2Z)-2-butenedioate (1:1), having the structure as represented in Formula I.

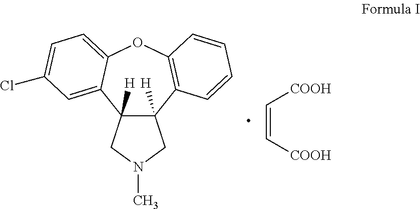

Asenapine maleate is marketed in the United States under the brand name SAPHRIS®, for the treatment of schizophrenia.

Processes for the preparation of asenapine maleate and intermediates thereof are disclosed in U.S. Pat. Nos. 4,145,434 and 7,872,147; PCT Publication Nos. WO 2009/008405, WO 2008/081010 and WO 2009/087058 and in Organic Process Research and Development, Vol. 12, p. 196-201 (2008), which are incorporated herein by reference.

U.S. Pat. No. 4,145,434 describes a process for the preparation of asenapine maleate which involves reducing the carbon-carbon double bond of the intermediate of Formula II

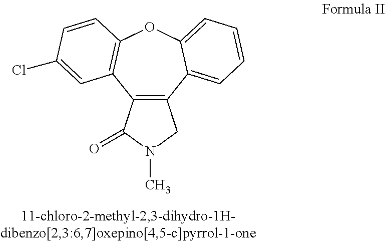 [SMILES: CN1CC2=C(C1=O)c1cc(Cl)ccc1Oc1ccccc12]

to obtain the intermediate of Formula III

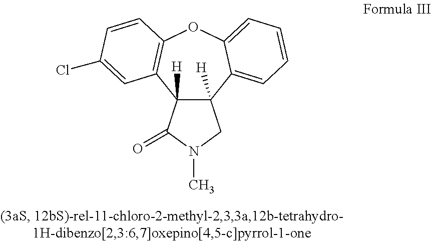

by a process involving the addition of a solution of an intermediate of Formula II in toluene to a suspension of magnesium in a mixture of toluene and methanol.

The magnesium-methanol process disclosed in U.S. Pat. No. 4,145,434 for the preparation of an intermediate of Formula III is not suitable for an industrial scale preparation due to its poor product selectivity and the associated safety concerns as detailed below:
Poor product selectivity as the desired trans-isomer and undesired cis-isomer are formed in an unfavorable ratio of about 1:4.
Poor reaction control, because the reaction between magnesium and methanol is heterogeneous and exothermic in nature. This limits the maximum scale at which one can safely operate the process and results in the formation of a significant amount of side products.
The use of carcinogenic dibromomethane for the activation of magnesium metal poses a health hazard.

Organic Process Research and Development, Vol. 12, p. 196-201 (2008), describes a dose-controlled reverse addition process wherein instead of adding an intermediate of Formula II to a suspension of magnesium in methanol and toluene, portions of the magnesium-methanol suspension are added to a solution of the intermediate of Formula II. Although the dose-controlled reverse-addition process helped in overcoming the drawbacks associated with poor reaction control, this process failed to provide any improvement in terms of product selectivity, and the desired trans-isomer and undesired cis-isomer still being formed in an unfavorable ratio.

Organic Process Research and Development, Vol. 12, p. 196-201 (2008), further describes that only magnesium in a combination with methanol was able to reduce the carbon-carbon double bond of the intermediate of Formula II. The carbon-carbon double bond of an intermediate of Formula II cannot be reduced using catalytic hydrogenation with other metal catalysts such as palladium, platinum, rhodium, ruthenium, iridium, zinc and lithium, using magnesium in ethanol, using magnesium in propanol, or by a Birch reduction.

In view of the drawbacks associated with the prior art processes, especially poor product selectivity and the inability of other metal catalysts and solvents or Birch reductions in reducing the double bond of the intermediate of Formula II, there exists a need in the art for a process which provides better product selectivity.

### SUMMARY OF THE INVENTION

The present inventors have developed an improved process for the preparation of the trans-intermediate of Formula III having better product selectivity. The process of the present invention involves preparation of the intermediate of Formula III by carrying out the reduction of the intermediate of Formula II using a magnesium-methanol-acetic acid mixture. The process of the present invention provides the intermediate of Formula III in 1:1 cis: trans ratio.

A first aspect of the present invention provides a process for the preparation of an intermediate of Formula III

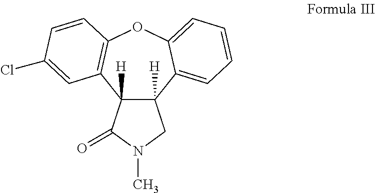

comprising reducing the intermediate of Formula II

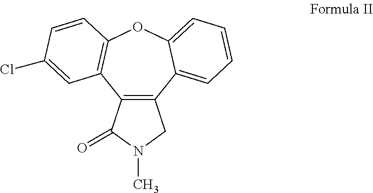 [SMILES: CN1CC2=C(C1=O)c1cc(Cl)ccc1Oc1ccccc12]

using a magnesium-methanol-acetic acid mixture.

A second aspect of the present invention provides a process for the preparation of asenapine maleate of Formula I

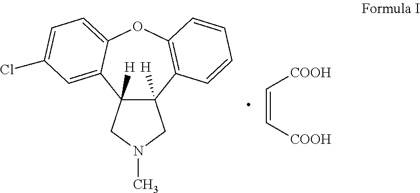

comprising the steps of:
i) reducing the intermediate of Formula II

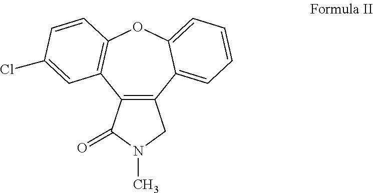 [SMILES: CN1CC2=C(C1=O)c1cc(Cl)ccc1Oc1ccccc12]

using a magnesium-methanol-acetic acid mixture to obtain the intermediate of Formula III;

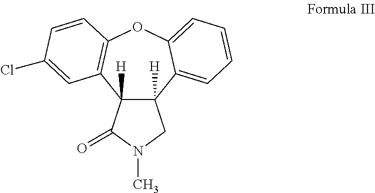

ii) reducing the carbonyl group of the intermediate of Formula III to obtain asenapine of Formula IV; and

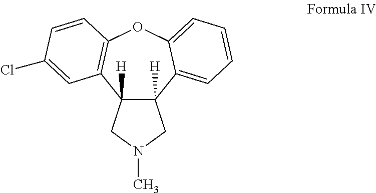

iii) converting asenapine of Formula IV to asenapine maleate of Formula I.

Other objects, features, advantages and aspects of the present invention will become apparent to those of ordinary skill in the art from the detailed description provided below.

### DETAILED DESCRIPTION OF THE INVENTION

The term “ambient temperature”, as used herein, includes temperature in the range of about 20° C. to about 35° C.

The intermediate of Formula II, to be used for the preparation of the intermediate of Formula III, may be prepared by the process disclosed in U.S. Pat. No. 4,145,434 which is incorporated herein by reference.

The conversion of the intermediate of Formula II into the intermediate of Formula III may be carried out by dissolving the intermediate of Formula II in a mixture of methanol and acetic acid. The reaction mixture may be heated to about 40° C. to 65° C. Magnesium metal turnings may be added portion-wise. The reaction mixture may be stirred for about 30 minutes to about 5 hours. The magnesium salts formed during the reaction and the un-reacted magnesium may be removed from the reaction mixture either by adding water and optionally adjusting the pH of the reaction mixture to about 1 to 2 by adding concentrated hydrochloric acid, or by filtration, followed by extraction with a solvent and removal of the solvent by distillation under reduced pressure to obtain a mixture of diastereomers. In embodiments involving removal of the magnesium salts and un-reacted magnesium by filtration, the resulting solid material may be further extracted with a solvent to extract the mixture of diastereomers from the solid material. The mixture of diastereomers may then be separated into cis- and trans-isomers by silica gel column chromatography using an ethyl acetate:hexane (30:70) mixture as the eluent.

The solvent to be used for carrying out the extraction may be selected from water-immiscible solvents selected from the group comprised of hydrocarbons, ethers, alkyl acetates or chlorinated hydrocarbons. Examples of hydrocarbons may include toluene, benzene or xylene. Examples of ethers may include diethyl ether, ethyl methyl ether or tetrahydrofuran. Examples of alkyl acetates may include ethyl acetate or di-isopropyl acetate. Examples of chlorinated hydrocarbons may include dichloromethane or chloroform.

The process of the present invention provides the intermediate of Formula III in a 1:1 cis: trans ratio.

The reduction of the carbonyl group of the intermediate of Formula III to obtain asenapine of Formula IV may be carried out using complex metal hydrides such as di-isobutylaluminum hydride, lithium borohydride or sodium trimethoxyborohydride. The reduction of the carbonyl group of the intermediate of Formula III may also be carried out using borane dimethyl sulphide. The reduction of the carbonyl group of the intermediate of Formula III may be carried out in an organic solvent selected from ethers or hydrocarbons. Examples of ethers may include diethyl ether, ethyl methyl ether, di-isopropyl ether, tetrahydrofuran or 1,4-dioxane. Examples of hydrocarbons may include benzene, toluene or xylenes.

In a preferred embodiment of the present invention, reduction of the carbonyl group may be carried out by adding a solution of borane dimethyl sulphide in tetrahydrofuran to a pre-heated solution of the intermediate of Formula III in tetrahydrofuran at a temperature of about 50° C. to about 80° C. in an inert atmosphere. The reaction mixture may be stirred for about 8 to about 16 hours. Dimethyl sulphide produced during the reaction may be slowly distilled-off from the reaction mixture. Fresh tetrahydrofuran may be added to compensate for the loss of tetrahydrofuran during distillation. An additional amount of borane dimethyl sulphide solution may be added and the reaction mixture may be stirred for about 1 hour to about 6 hours for completion of the reaction. Alcohol selected from the group comprising methanol, ethanol or propanol may be added. The contents may be stirred for about 5 to about 30 minutes followed by the addition of a mixture of sulphuric acid and water. The reaction mixture may be stirred at about 60° C. to about 90° C. for about 4 to about 10 hours, cooled, then extracted with a solvent selected from hydrocarbon solvents such as benzene, toluene, xylenes, monochlorobenzene or 1,2-dichlorobenzene. Water may be added followed by the slow addition of an ammonia solution in a period of about 5 to about 30 minutes. Asenapine of Formula IV may be extracted from the reaction mixture by adding a solvent selected from hydrocarbon solvents such as benzene, toluene, xylenes, monochlorobenzene or 1,2-dichlorobenzene followed by drying.

Drying may be accomplished by any suitable method such as air drying, drying under reduced pressure, vacuum tray drying or a combination thereof. Drying may be carried out at ambient temperature to a temperature of about 80° C.

Conversion of asenapine of Formula IV into asenapine maleate of Formula I may be carried out by conventional methods such as the method described in U.S. Pat. No. 4,145,434.

In the foregoing section, embodiments are described by way of examples to illustrate the process of invention. However, these are not intended in any way to limit the scope of the present invention. Variants of the examples evident to persons ordinarily skilled in the art are within the scope of the present invention.

### EXAMPLES

### Comparative Example

### Preparation of Trans-11-Chloro-2-Methyl-2,3,3a,12b-Tetrahydro-1H-Dibenzo[2,3:6,7]Oxepino[4,5-C]Pyrrol-1-One (Formula III)

7 g of 11 -chloro-2-methyl-2,3 -dihydro-1H-dibenzo [2,3:6,7]oxepino[4,5-c]pyrrol-1-one was suspended in methanol (50 mL). Magnesium metal turnings (10 g) were added. The reaction mixture was slowly heated to reflux temperature. Brisk effervescence was observed. The reaction mixture was cooled to control the reaction, again refluxed for about 2 hours under controlled conditions, diluted with methanol (30 mL), further refluxed for about 30 minutes and cooled to ambient temperature. Methanol (150 mL) was added. The pH was adjusted to about 1 to 2 by adding concentrated hydrochloric acid, and a clear solution was obtained. The solution was extracted with ethyl acetate (3×100 mL) and the combined ethyl acetate layers were washed with water (3×50 mL). Ethyl acetate was removed by distillation under reduced pressure to obtain a mixture of two isomers as a brown oil (4.6 g). The mixture of isomers was separated into cis- and trans-isomers using silica gel column chromatography eluting with ethyl acetate: hexane.

trans-isomer: 0.65 mg
cis-isomer: 3.5 g

### WORKING EXAMPLES

### Example 1

### Preparation of Trans-11-Chloro-2-Methyl-2,3,3a,12b-Tetrahydro-1H-Dibenzo[2,3:6,7]Oxepino[4,5-C]Pyrrol-1-One (Formula III)

2 g of 11-chloro-2-methyl-2,3-dihydro-1H-dibenzo[2,3:6,7]oxepino[4,5-c]pyrrol-1-one was dissolved in a mixture of methanol (60 mL) and acetic acid (20 mL). The reaction mixture was heated to about 53° C. Magnesium metal turnings (2.0 g) were added portion-wise. The reaction mixture was stirred for about 1 hour, filtered and washed with methanol (100 mL). Methanol was removed by distillation from the filtrate to obtain a white solid (16 g). The white solid was dissolved in dichloromethane (200 mL) and washed with water (2×500 mL). The solid obtained during filtration was also dissolved in water (100 mL) and the aqueous layer was extracted with dichloromethane (50 mL). The two dichloromethane solutions were combined. Dichloromethane was removed by distillation under reduced pressure to obtain a mixture of two isomers as an oily brown compound (2 g). The mixture of isomers was separated into cis- and trans-isomers using silica gel column chromatography eluting with ethyl acetate:hexane (30:70) mixture.

trans-isomer: 0.7 g
cis-isomer: 0.7 g

### Example 2

### Preparation of Trans-11-Chloro-2-Methyl-2,3,3a,12b-Tetrahydro-1H- Dibenzo[2,3:6,7]Oxepino[4,5-C]Pyrrol-1-One (Formula III)

2 g of 11-chloro-2-methyl-2,3-dihydro-1H-dibenzo[2,3:6,7]oxepino[4,5-c]pyrrol-1-one was dissolved in a mixture of methanol (60 mL) and acetic acid (20 mL). The reaction mixture was heated to about 50° C. Magnesium metal turnings (2.38 g) were added portion-wise at about 45° C. to about 65° C. The reaction mixture was stirred at ambient temperature for about 2 hours. Water (80 mL) was added. The pH of the reaction mixture was adjusted to 1 by adding concentrated hydrochloric acid. The reaction mixture was extracted with ethyl acetate (150 mL) and washed with water (3×200 mL). Ethyl acetate was distilled-off to obtain a mixture of two isomers as an oily brown compound (2 g). The mixture of isomers was separated into cis- and trans-isomers using silica gel column chromatography eluting with ethyl acetate:hexane (30:70) mixture.

trans-isomer: 0.7 g
cis-isomer: 0.8 g

### Example 3

### Preparation of Asenapine [Formula IV]

A 2M solution of borane dimethyl sulphide in tetrahydrofuran (128 mL) was added drop-wise to a pre-heated solution (heated to about 64° C.) of trans-(3a,12b)-11-chloro-2-methyl-2,3,3a,12b-tetrahydro-1H-dibenzo[2,3:6,7]oxepino[4,5-c]pyrrol-1-one (30 g) in tetrahydrofuran (300 mL) at about 64° C. under nitrogen flow. The reaction was allowed to proceed for about 12 hours. Dimethyl sulphide produced during the reaction was slowly removed by distillation from the reaction mixture and fresh tetrahydrofuran was added. Borane dimethylsulphide in tetrahydrofuran 2M solution (24 mL) was added and the reaction mixture was stirred for about 3 hours. Tetrahydrofuran was distilled-off under reduced pressure. Methanol (250 mL) was added to the residue and the reaction mixture was stirred for 15 minutes. A sulphuric acid:water mixture (75 mL:500 mL) was added over about 5 minutes. The reaction mixture was stirred at about 80° C. for about 7 hours, cooled to about 50° C. and washed with toluene (2×200 mL). The layers were separated. The aqueous layer was cooled to about 0° C. to 5° C., and the crystallized salt was filtered, washed with cold water (100 mL) and dried in air at about 45° C. for about 15 hours.

29 g of the air-dried material was suspended in water (150 mL). An ammonia solution was added slowly over about 10 minutes. Asenapine was extracted from the reaction mixture by adding toluene (2×100 mL), washing with water (100 mL), then removing toluene by distillation under reduced pressure.

Yield: 22.2 g

We claim:

1. A process for the preparation of the intermediate of Formula III 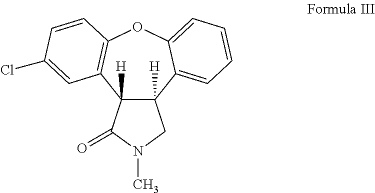
comprising reducing the intermediate of Formula II 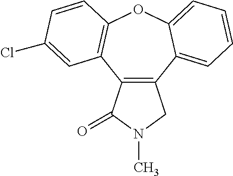 [SMILES: CN1CC2=C(C1=O)c1cc(Cl)ccc1Oc1ccccc12]
using a magnesium-methanol-acetic acid mixture.

2. A process for the preparation of asenapine maleate of Formula I 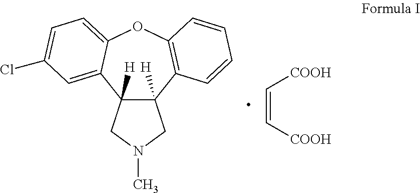
comprising the steps of:
i) reducing the intermediate of Formula II 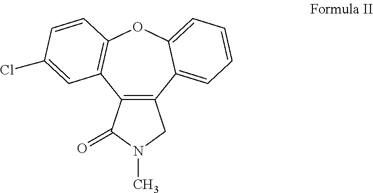 [SMILES: CN1CC2=C(C1=O)c1cc(Cl)ccc1Oc1ccccc12]
using a magnesium-methanol-acetic acid mixture to obtain the intermediate of Formula III; 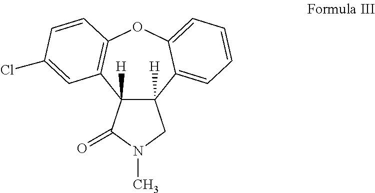
ii) reducing the carbonyl group of the intermediate of Formula III to obtain asenapine of Formula IV; and 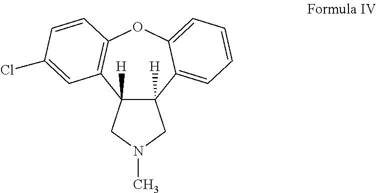
iii) converting asenapine of Formula IV to asenapine maleate of Formula I.

3. The process according to claim 1 or 2, wherein the reduction of the intermediate of Formula II is carried out at a temperature of about 40° C. to about 65° C.

4. The process according to claim 1 or 2, wherein the reduction of the intermediate of Formula II is carried out in about 30 minutes to about 5 hours.

5. The process according to claim 2, wherein the reduction of the carbonyl group of the intermediate of Formula III is carried out using complex metal hydrides selected from di-isobutylaluminum hydride, lithium borohydride or sodium trimethoxyborohydride.

6. The process according to claim 2, wherein the reduction of the carbonyl group of the intermediate of Formula III is carried out using borane dimethyl sulphide.

7. The process according to claim 2, wherein the reduction of the carbonyl group of the intermediate of Formula III is carried out in an organic solvent selected from ethers and hydrocarbons.

8. The process according to claim 2, wherein the reduction of the carbonyl group of the intermediate of Formula III is carried out at about 50° C. to about 80° C.

9. The process according to claim 2, wherein the reduction of the carbonyl group of the intermediate of Formula III is carried out in about 8 to about 16 hours.

10. The process according to claim 1 or 2, wherein the intermediate of Formula III is obtained in 1:1 cis:trans ratio.
